(19) United States
(12) Patent Application Publication
(10) Pub. No.: US 2006/0058855 A1
(43) Pub. Date: Mar. 16, 2006

(54) Deep brain stimulation

(76) Inventor: Steven Streatfield Gill, Bristol (GB)

Correspondence Address: Kenneth I. Kohn;Kohn & Associates, PLLC, Suite 410, 30500 Northwestern Hwy., Farmington Hills, MI, 48334, US

(21) Appl. No.: 11/112,301

(22) Filed: Apr. 22, 2005

(30) Foreign Application Priority Data
Apr. 23, 2004 (GB) 0409109.6

(51) Int. Cl. A61N 1/18

(52) U.S. Cl. 607/45

(57) ABSTRACT

A method for treating essential tremor comprising the step of applying deep brain stimulation to the ascending dentate/interpositus-ventral intermedius fibres of the brain at a location remote from the ventral intermedius nucleus of the thalamus. A method for identifying an area of a patient's brain to be targeted with deep brain stimulation for the treatment of essential tremor comprising the step of using a scan of a patient's brain to identify a target area in relation to the subthalmic nucleus and the red nucleus. A method of treating essential tremor by using deep brain stimulation. A method of treating essential tumor by using a DBS electrode targeted to the dentate/interpositus-ventral intermedius fibres. A kit used in treating essential tremor.

### CROSS-REFERENCE TO RELATED APPLICATIONS

This application claims the benefit of priority to Patent Application Number GB 0409109.6, filed Apr. 23, 2004, which is incorporated herein by reference in its entirety.

### BACKGROUND OF THE INVENTION

1. Technical Field

The present invention relates to a method for treating essential tremor (ET) using deep brain stimulation (DBS), and to a method of identifying an area of the brain to be targeted by DBS in the treatment of ET. It further relates to the use of DBS in the treatment of ET.

2. Background Art

ET is a common movement disorder affecting between 300 to 415 people per 100,000. The incidence of new cases increases with age and it is known to affect both men and women equally. ET has an autosomal dominant inheritance with variable clinical expression and almost complete penetrance by the age of 65 years.

The etiology of ET is poorly understood. Although no morphological changes have been identified, it has been attributed to a functional disturbance in the inferior olivary nucleus, where abnormally synchronised 4-12 Hz oscillations occur. These probably result from excessive electrotonic coupling between dendrites of the inferior olivary neurons via GABA mediated gap junctions. The abnormal oscillations are transmitted via the Purkinje cells and Dentate/Interpositus nucleus and then distributed to thalamocortical and brainstem nuclei. Clinical case reports of infarcts or lesions involving these pathways in ET patients have been shown to arrest tremor.

The inferior olive is thought to play an important role as a teacher of the cerebellum in adjusting or modulating planned movements during their execution, in response to unconditioned afferent information. It achieves this by modulating cerebellar return to the motor cortex via the Purkinje cells. In ET patients, if there is an excessive recruitment of inferior olive neurons in response to afferent information, and the neurons oscillate synchronously in the 4-12 Hz range, then there will be a potent effect on motor performance which will be manifested as tremor.

Drug treatment is effective in only 50% of patients and those who are refractory may be offered stereotactic surgery. Typically the Ventralis Intermedius (Vim) nucleus of the thalamus is the target of choice and lesioning is reported to provide good contralateral tremor suppression. However recurrence may occur within weeks or years and long-term studies show that significant tremor persists in 17-32% of cases. Bilateral lesions are associated with significant complications including permanent speech impairment in over 25% and memory and language dysfunction in over 50% of cases.

Clinical studies suggest that DBS of Vim is as effective as lesioning in controlling tremor, but is likewise associated with side effects, particularly when carried out bilaterally with 30-50% patients suffering from dysarthria and dysequilibrium. However the adverse effects associated with DBS are generally reversible by adjusting the stimulation parameters, though this may be that the expense of satisfactory tremor control. Patients treated with DBS are also reported to develop tolerance (habituation) to stimulation, despite increasing its amplitude. Patients are advised to turn the stimulators “off” at night and take stimulation holidays for weeks, in order to prevent tissue habituation.

In 1965, Mundinger reported good results by making large lesions in the subthalamic region for control of ET. Subsequently, in 1969, Bertrand defined an area where the mere impact of the tip of a small probe caused abrupt and total cessation of tremor. This area was in the region of the “prelemniscal radiation” (most posterior and inferior portion of the zona incerta (ZI) or posterodorsal to the subthalamic nucleus, corresponding to coronal slice FP 7.0 on the Schaltenbrand atlas). He attributed his findings to lesioning the ascending fibres from the upper mesencephalic reticular substance, pallido-thalamic and pallido-tegmental fibres. We now know that this area also carries the dentate/interpositus-thalamic fibres on way to the Vim nucleus of the thalamus. Subsequently, long term follow up studies of lesions involving the ventral thalamus and subthalamic region showed improvement in ET, with a low complication rate. Hypotonia and gait disturbance were observed in 5%, and speech disturbance in 1%. Kitagawa et al have recently reported two cases of severe proximal ET with good results, controlled by stimulating the subthalamic region including the ZI.

Muarta et al., describes applying stimulation to the “prelemniscal radiation” to treat proximal tremor (Muarta et al., (2003) J. Neurosurgery 99, 708-715). Stimulating this area in general is likely to cause stimulation of the lemniscus which would result in side effects such as tingling and numbness in the limbs. Further, Muarta et al., only describes unilateral treatment. Bilateral treatment is not normally given for limb tremor as it may result in side effects like speech disturbance. It would be advantageous to identify a target site for treatment of tremor that could be used bilaterally, that is remote from the lemniscus and which produces reduced or no side effects.

There is a need for an effective treatment for ET, particular one which does not cause the side effects associated with the treatments presently employed. The inventor has found that ET can be treated by using DBS on a part of the brain that has not previously been targeted. This treatment avoids at least some of the problems associated with some of the prior art methods.

### SUMMARY OF THE INVENTION

According to the present invention, there is provided a method for treating essential tremor comprising the step of applying deep brain stimulation to dentate/interpositus-ventral intermedius fibres of the brain at a location remote from the ventral intermedius nucleus. The present invention also provides a method for identifying an area of a patient's brain to be targeted with deep brain stimulation for the treatment of essential tremor comprising the step of using a scan of a patient's brain to identify a target area in relation to the subthalmic nucleus and the red nucleus. Further, the present invention provides a method of treating essential tremor by using deep brain stimulation of the dentate/interpositus-ventral intermedius fibres of the brain at a location remote from the ventral intermediate nucleus of the thalamus in the treatment of essential tremor. Additionally, the present invention provides a method of treating essential tumor by using a DBS electrode targeted to the dentate/interpositus-ventral intermedius fibres at a location remote from the ventral intermedius nucleus of the thalamus in the preparation of a component for the treatment of essential tremor. The present invention further provides a kit for use in treating essential tremor.

### DESCRIPTION OF THE DRAWINGS

Other advantages of the present invention are readily appreciated as the same becomes better understood by reference to the following detailed description when considered in connection with the accompanying drawings wherein:

FIG. 1 is a high-resolution axial T2-weighted MR images showing the delineated subthalamic nucleus (STN), red nucleus (RN) and the mamillo-thalamic tracts (MT);

FIG. 2 is a pre-operative high-resolution coronal T2-weighted MR images showing the outlined head of the caudate nucleus (CD), thalamus (TH), subthalamic nucleus (STN) and substantia nigra (SN);

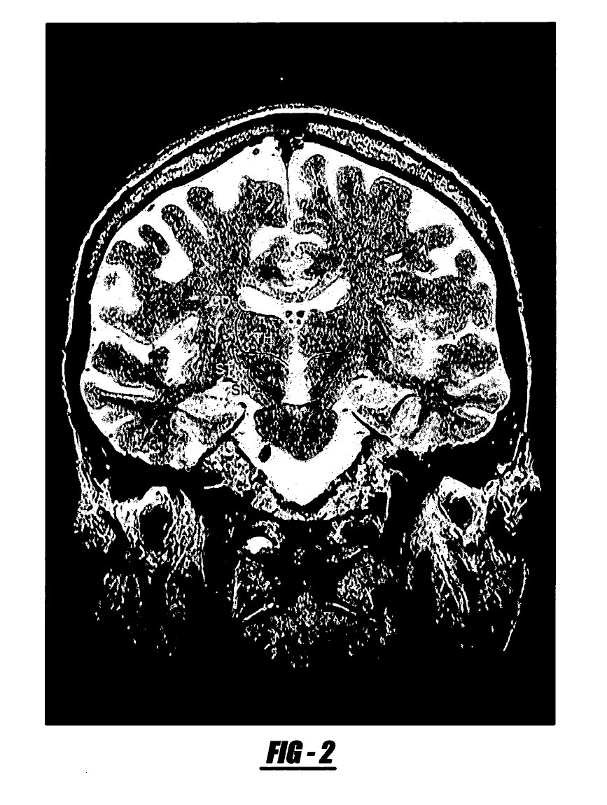

FIG. 3a is a line diagram drawn to scale, showing the peroperative position of the guide tube and stylette to the planned target in the subthalamic region;

FIG 3b is a line diagram drawn to the same scale as in 3a, the stylette has been removed and replaced with the DBS electrode, wherein the position of the DBS electrode and its contacts to the planned target in the subthalamic region is shown;

FIG. 4 is a per-operative inverted axial T2 weighted image verifying the position of the radio-opaque stylettes (red arrows) within the planned target, wherein per-operative images are obtained in the same slice configuration as the pre-operative planning images;

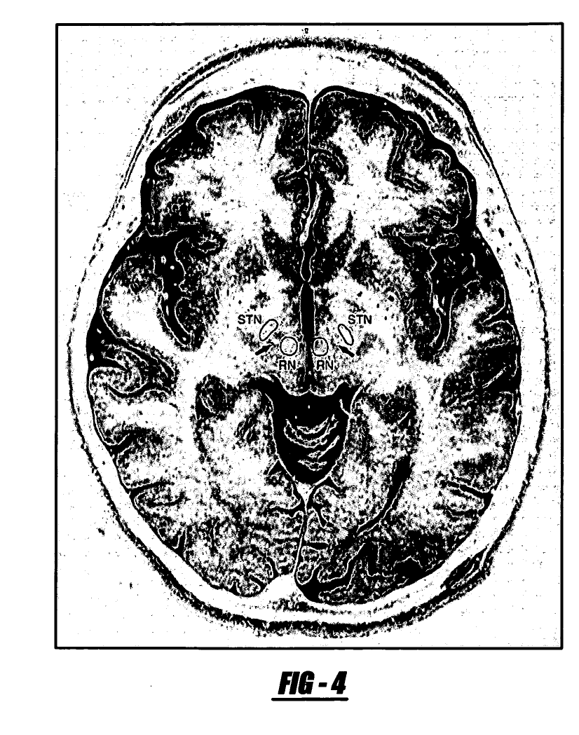

FIG. 5 is a per-operative inverted coronal T2 weighted image verifying the position of the radio-opaque stylettes within the planned target, wherein per-operative images are obtained in the same slice configuration as the pre-operative planning images;

FIG. 6 shows mean clinical tremor score (Total, Part A, Part B, Part C) at baseline and at 12 month evaluation with DBS “ON” and “OFF”, wherein the data is presented as mean +standard deviation (SD) DBS indicates deep brain stimulation;

FIG. 7 is a schematic diagram showing the position of the deep brain stimulation electrode in relation to the path of the Cerebello-Vim fibres from the Dentate and Interpositus nucleus on the left side. The electrode is positioned where these fibres are concentrated together in the subthalamic region before “fanning out” to the large body of the Vim nucleus above; and

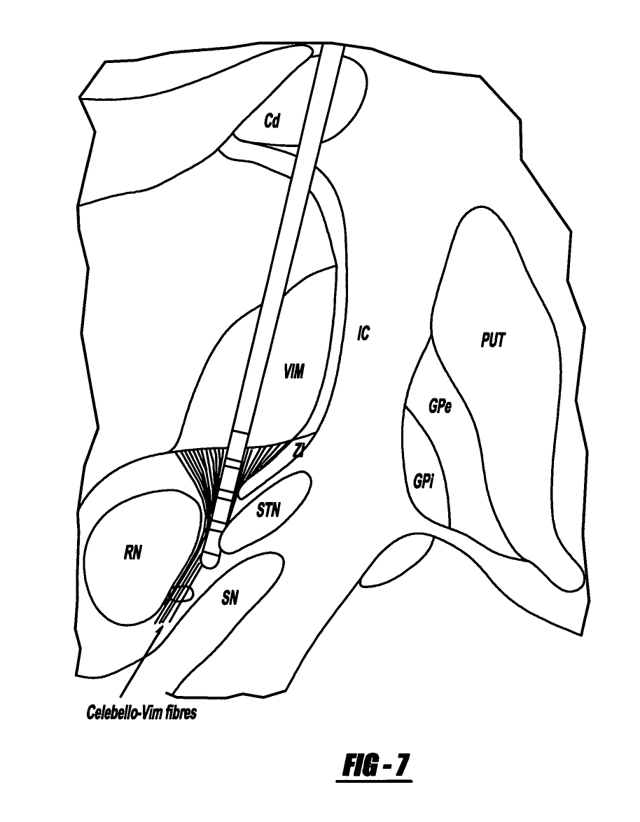

FIG. 8 is a schematic diagram showing a patient who has two electrodes implanted into the brain, and a pulse generator implanted under the skin, in accordance with a method of the invention (Key: Cd (Caudate Nucleus), VIM (Ventralis Intermedius), ZI (Zona Incerta), STN (Subthalamic nucleus), RN (Red nucleus), SN (Substantia Nigra), IC (Internal capsule), PUT (Putamen), GPe (Globus pallidus externus), Gpi (Globus pallidus internus)).

### DETAILED DESCRIPTION OF THE INVENTION

Essential tremor is a chronic neurological disease which is characterised by involuntary, rhythmical tremor of an area of the body, such as arms, hands, legs, head, chin, and voice. Hand tremor is seen most commonly, and is usually bilateral (affecting both hands).

The ascending dentate/interpositus-ventral intermedius (Vim) fibres run from the dentate and interpositus nuclei to the ventral intermedius nucleus in the thalamus. The terms ascending dentate/interpositus-ventral intermedius fibres is well known to those skilled in the art.

In contrast to the known methods of treating ET, the method according to the invention targets the -fibres conveying the abnormal oscillations which produce the motor symptoms of ET as they pass from the deep cerebellar nuclei (the dentate and interpositus nuclei) to the thalamus, and in particular to the Vim nucleus.

Patients treated with the method of the invention do not develop the tolerance normally associated with DBS of the thalamic nuclei used in the treatment of ET. Tolerance is thought to develop because of adaptations that occur within the thalamic nuclei which also contain large numbers of inhibitory interneurons which are stimulated to the same degree as the thalamocortical neurons.

Further, patients treated with the method of the invention do not suffer from the side effects usually associated with DBS treatment of the thalamic nuclei. In particular, those side effects include speech disturbance and dysequilibrium. In fact, the inventor has noted an improvement in the speech of a patient who had dysarthria prior to treatment with the method of the invention. The reduction in side effects is likely to be brought about by accurate targeting of the dentate-thalamic fibres. The Vim nucleus is wedge-shaped, and is difficult to target in a field of stimulation that is typically oval or spherical in shape. As a result, current spreads to areas beyond the Vim nucleus, and causes side effects.

Deep brain stimulation is application of an electric field to an area of the brain. Deep brain stimulation may be applied by any method known to one skilled in the art.

The step of applying DBS preferably comprises stimulating the dentate/interpositus—Vim fibres with an electrical field that is sufficiently remote from the sensory thalamus to avoid stimulation of the sensory thalamus.

The application of DBS preferably comprises stimulating the dentate/interpositus—Vim fibres with an electric field that is remote from synaptic connections of the ventral intermedius nucleus of the thalamus.

The area to which DBS is applied may preferably be identified on the Shaltenbrand Bailey Stereotactic Atlas of the Human Brain, Axial plate 56 LXXVIII H. v 3.5 mm positioned 6.5 mm posterior to the intercommisural point and 11.5 mm lateral to the anterior/posterior commisural line.

Preferably the step of applying DBS further comprises the step of introducing an electrode into the brain, such that the electrode is in contact with the dentate/interpositus-ventral intermedius fibres. Any known DBS electrode may be used. The term “DBS electrode” refers to any electrical conducting lead for enabling the production of an electric field at a desired site suitable for use in DBS. Such electrodes are well known to those skilled in the art, for example those supplied by Medtronic, Inc., Minneapolis, Minn.

In addition, the method preferably further comprises connecting the electrode to an electricity supply, in particular to a pulse generator. Any :known pulse generator may -be used, for example, those supplied by Medtronic, Inc., Minneapolis, Minn.

During DBS the electrode is used to produce an electric field at a desired target site. The electrode has a proximal end which is connected to the pulse generator. The proximal end is preferably connected to the pulse generator via an insulated wire. The DBS electrode also preferably has a distal end which is positioned at the target site.

The step of connecting the electrode to a pulse generator preferably includes providing the electrode on a lead having at least one conductor, and connecting the lead to the pulse generator; the method further comprising implanting the pulse generator in the body of the patient wherein the step of implanting the pulse generator the body of the patient comprises implanting the pulse generator in one of a cranial region or a pectoral region.

The electrode may be located at the target site by any known method. The method of the invention may be carried out, for example on an awake patient using micro electrode recording (MER) techniques, or on an anaésthetised patient using MRI scanning. Such surgical methods are well known to those skilled in the art, any appropriate surgical method may be used.

The DBS is preferably applied at a mean voltage between 1.0 and 2.5V, more preferably at between 1.2 and 2.3V, even more preferably at between 1.6 and 2.0V, and most preferably at 1.8V.

The inventor attributes the success in treating ET, even at low voltages to the fact that the target area, namely the dentate/interpositus-ventral intermedius fibres are confined to a small volume within the subthalamic region, and can be targeted accurately.

In addition, neuronal axons, as found in the fibres, are approximately ten times more readily excitable than nuclei. Hence, stimulating the axons has a much more potent effect, allowing lower voltages to be used.

DBS can be applied monolaterally or bilaterally, but is preferably applied bilaterally. Bilateral means that DBS is applied to both hemispheres of the brain. DBS is preferably applied bilaterally because ET usually affects both sides of the body, and is controlled by both sides of the brain.

Either a mono-polar or bi-polar electric field may be used. Preferably a mono-polar electric field is used.

Depending on the way the electrode is connected to the pulse generator, it is possible to create a mono-polar or a bi-polar electric field. Altering the connections of an electrode to a pulse generator is well known to those skilled in the art. In particular, the technical manual for Medtronic's DBS leads 3389 and 3387 clearly discusses changing electrical connections at the proximal end of an electrode to change the electric field generated at the distal end of the electrode.

In the method of the invention, the deep brain stimulation is preferably applied continually.

Continuous application means that pulses of DBS are applied repeatedly without any significant lapses between pulses.

DBS is preferably applied at a frequency of between 100 Hz and 200 Hz. More preferably it is applied at between 120 and 190 Hz, and even more preferably at between 130 and 180 Hz.

The invention also provides a method for identifying an area of a patient's brain to be targeted with deep brain stimulation for the treatment of essential tremor, comprising the step of using a scan of a patient's brain to identify a target area in relation to the subthalamic nucleus and the red nucleus.

This method provides an initial non-surgical step which is preferably taken in order to subsequently apply DBS to the target area identified.

The target area can be readily identified using, for example MRI imaging, because both the subthalamic nucleus and red nucleus can be identified on MRI images. Other sites, such as the Vim nucleus cannot be identified on MRI images. Identifying target sites using, for example, MRI imaging, allows surgery to access the target site, following identification, to be carried out under general anaesthetic. This is particularly useful in patients suffering from extreme tremors. Further surgery is technically more straightforward.

The target area is preferably further defined in relation to the zona incerta, the ventral thalamus and the medial lemniscus. More preferably, the target area is medial to the posterior dorsal third of the subthalamic nucleus.

The target area encompasses dentate/interpositus-Ventral Intermedius fibres. The target area can preferably be identified on the Shaltenbrand Bailey Stereotactic Atlas of the Human Brain, Axial plate 56 LXXVIII H. v-3.5 mm positioned 6.5 mm posterior to the intercommisural point and 11.5 mm lateral to the anterior/posterior commisural line.

The scan can be any known scan which can be used to identify the target area. Preferably the scan is an MR scan. More preferably the scan is a T₂ weighted MR scan.

Preferably the method of identifying a target area according to the invention is combined with the method of treating essential tremor according to the invention.

Further provided by the invention is the use of deep brain stimulation of dentate-thalamic fibres in the treatment of essential tremor. Preferably the dentate-thalamic fibres are dentate/interpositus-ventral intermedius fibres. Also provided is the use of a DBS electrode targeted to the dentate-thalamic fibres in the preparation of a component for the treatment of essential tremor. Preferably the DBS electrode is used at a location remote from the ventral intermedius nucleus of the thalamus.

The invention also provides a kit for use in treating essential tremor comprising a DBS electrode and instructions for how to identify the dentate-thalamic fibres. Preferably the kit also comprises instructions for how to position the DBS electrode during treatment. The electrode preferably has a proximal end for connection to an electricity supply, and a distal end, which, in use, is positioned in contact with the dentate-thalamic fibres.

The invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLES

Materials and Methods

Demographics

Four patients (3 female; 1 male) seen in the clinic with functionally disabling essential tremor (postural or intention tremor of the hands and forearm or an isolated head tremor without evidence of dystonia, and also absence of other neurologic signs except cogwheeling), despite maximum pharmacologic therapy (propranolol up to 320mg and primidone up to 750 mg) were considered candidates for surgery and were included in this pilot study. They had an average age of 66.8±8.5 years. The average duration of the disease for the women was 10.3±1.5 years. The sole male patient had ET for 38 years. A positive family history was seen in all patients. All patients gave fully informed consent and were aware of the potential risks of stereotactic surgery.

Clinical Evaluation

All patents were assessed using the Fahn-Tolosa-Marin Tremor Rating scale. This is divided into Parts A, B and C. Part A (Item 1 to 9) quantifies the tremor at rest, with posture holding, and with action and intention manoeuvres to the various body parts. This is rated on a 5 point scale {Grade 0=No tremor, Grade 1=Slight tremor (amplitude<0.5), May be intermittent. Grade 2=Moderate tremor (amplitude 0.5-1 cm), May be intermittent. Grade 3=Marked tremor (amplitude 1-2 cm). and Grade 4=Severe tremor (amplitude>2cm)}. Part B (Item 10 to 14) relates to action, tremors of the upper extremities, particularly writing and pouring liquids. Part C (Item 15 to 21) assesses functional disability with activities of daily living (Eating solids, Drinking liquids, Hygiene, Dressing, Writing and Working). Voice tremor was evaluated by listening to the patient talk, and the ability to utter a single sound as “aaahhh” and hold it for as long as possible. Evaluations were performed preoperatively and at 12 months postoperatively by a Specialist Movement disorder nurse. Preoperative assessments were performed with patients “off”-medication (propranolol and primidone) for 12 hours overnight. In the postoperative period they were assessed in two states: with the stimulator switched “off” for 3 hours and subsequently with the stimulator switched “on”.

MR Imaging and Target Planning

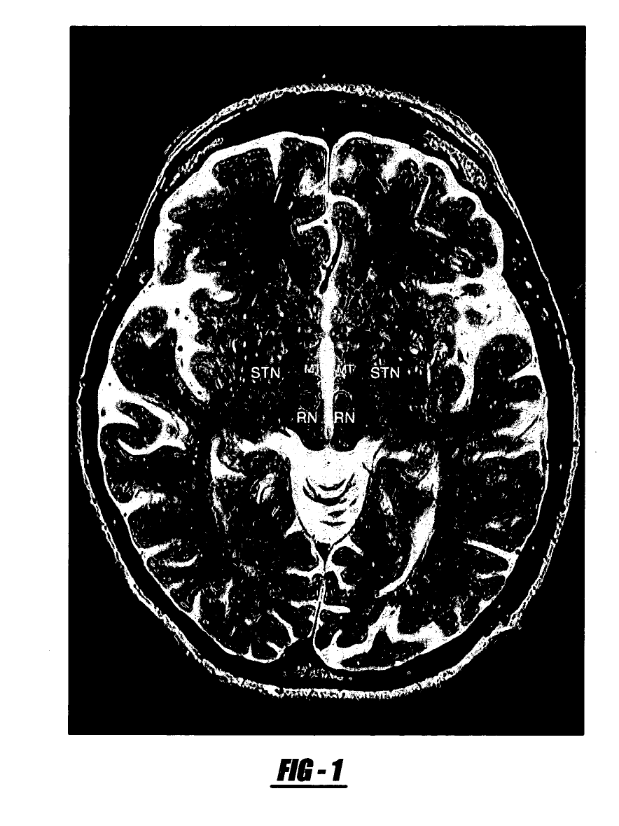

Ethical committee approval was obtained to perform stereotactic procedures under general anesthesia using implantable guide tubes to deliver the electrodes. The guide tube is an in-house investigational device manufactured by Ansamed Ltd; Rosscommon, Ireland. Under general anesthesia, a modified Leksell stereotactic frame (Elekta Instrument AB, Stockholm) was affixed parallel to the orbito-meatal plane. Patients then underwent high resolution MRI T2 scan sequences (1.5 Tesla TR 2,500, TE 150, TSE 11, NSA 12) to define the subthalamic nucleus (STN) and red nucleus. The anterior and posterior commissures (AC and PC) were identified in a mid-sagittal planning scan. Axial images 2 mm thick were acquired parallel to the AC-PC plane and coronal images orthogonal to these then obtained. Magnified hard copies of the T2 scans were obtained and overlaid on to inverted T2 images, further to enhance definition of the STN and surrounding structures. The boundary of the STN, red nucleus and related structures were outlined and a three-dimensional volume was created by cross correlating the boundaries on the axial and coronal images (FIG. 1,2).

The target area in the subthalamic region was then defined in relation to the STN, ZI, ventral thalamus, red nucleus and the medial lemniscus by using the Schaltenbrand atlas as a visual guide. The target area was medial to the posterior dorsal ⅓^{rd} of the STN, an area encompassing the ascending dentate/interpositus-Vim fibres and part of the ZI. The trajectory was planned, traversing the target, such that the 3^{rd} proximal contact on the DBS electrode (Contact 2 and 6) would be positioned at the planned target site and the distal end of the electrode deeper in the subthalamic region.

Surgery and Target Verification

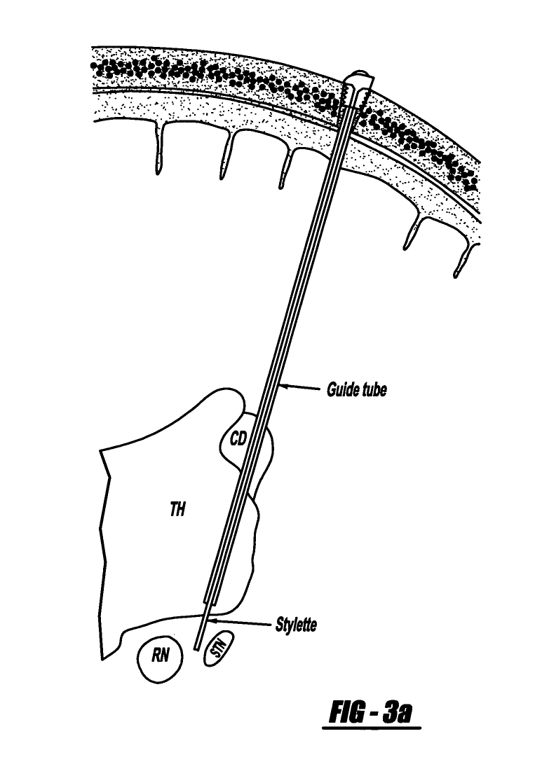

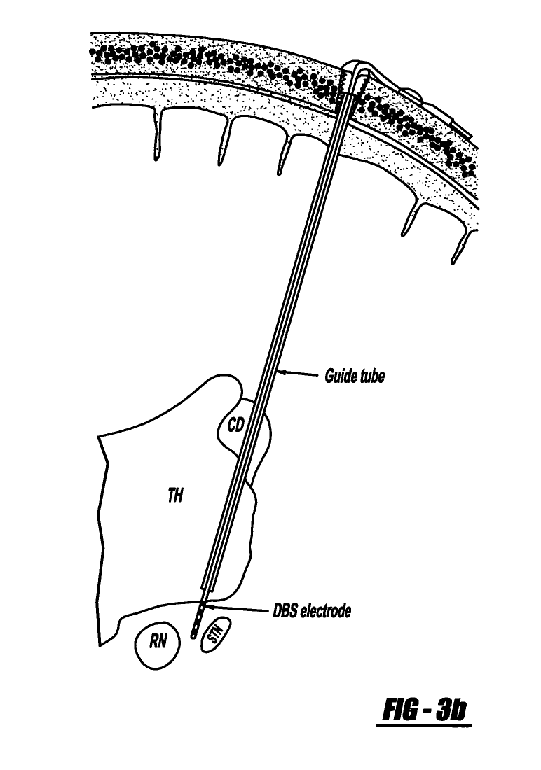

Surgery was performed under general anesthesia in semi-sitting position, such that the frontal burr holes were uppermost. This ensured that with constant saline irrigation and avoiding air entry, brain shift would be minimised. A probe was inserted to the target, and over this a plastic guide tube was advanced so that its distal end was short of the target by several millimetres. The proximal end of the guide tube, which is in the form of a hub was bonded within the burr hole with acrylic cement. The probe was then withdrawn and replaced with a plastic stylette (In-house investigational device, manufactured by Ansamed Ltd; Rosscommon, Ireland.) cut to an appropriate length, such that its distal end traversed the target to a planned position in the subthalamic region (FIG. 3a). This procedure was carried out bilaterally. Patients then underwent perop MR scans to verify the position of the plastic stylettes relative to the planned target (FIG. 3,4,5). Upon confirmation of satisfactory placement, the patient was returned to the operating theatre and the frame was removed. The plastic stylettes were removed and replaced with DBS leads (model 3387 and 3389 DBS leads, Medtronic Inc., Minneapolis) (FIG. 3b). The leads were secured to the skull with mini-plate and screws, and then connected to extension cables and the DBS pulse generator (Kinetra, Medtronic Inc., Minneapolis). The pulse generator was implanted in a subcutaneous pocket below the clavicle. The whole procedure typically took 3½-4 hours, including peroperative imaging and implantation of the DBS device.

Postoperative Management

The Kinetra generator was switched on immediately following surgery, and in the following days it was programmed by the to optimise tremor control. Movement disorder nurse, who optimised tremor control after programming patients through all the four DBS contacts. The specialist nurse was however blinded to the optimal planned DBS contact. Patients were advised to stop their anti-tremor medications gradually over the coming weeks.

Statistical Analysis

The primary efficacy was analysed using the paired Wilcoxon signed-rank and sign test. The test of significance was applied to the scores of the affected extremity, functional activities of upper limb and also the activities of daily living (ADL).

Results

Part A Score

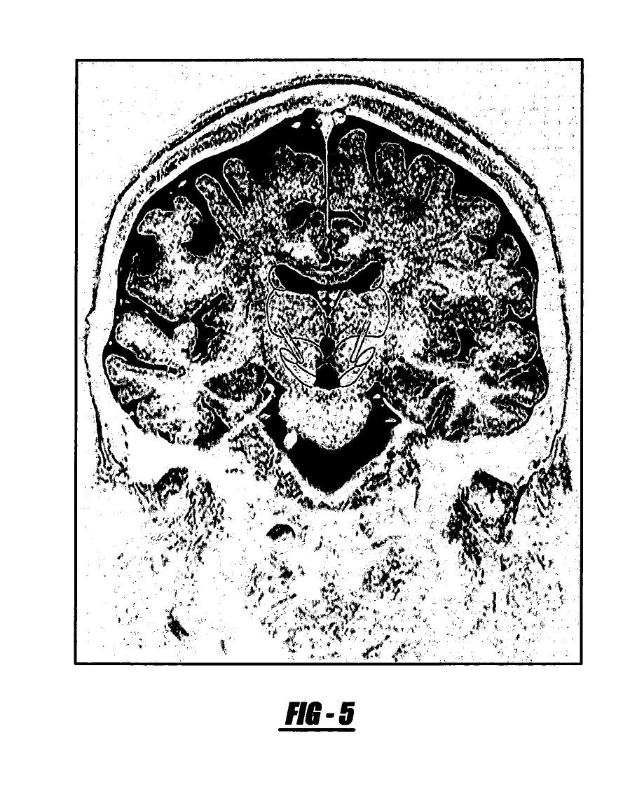

The total tremor score following surgery at 12-months improved by 80.1% (baseline mean score of 63±15.1 to 11.8±3.9 at 12 months). The Part A score (Item 1-9) improved by 84.2% (baseline mean score of 19±4.4 to 3.0±1.2 at 12 months) (FIG. 5 6). All patients had severe tremor in both the upper limbs (mean postural 3.0±0.9, mean action 3.4±0.7), though one patient also had rest tremor with no bradykinesia or rigidity. Following DBS implantation, tremor was completely arrested in ⅝ upper limbs, and a Grade 1 tremor was seen in the remainder. This was reflected in an overall improvement in the combined posture and action component tremor scores by 84.4% (mean baseline 3.2±0.8 to 0.5±0.-5 at 12 months, p value<0.0001) (Table. 1). A Grade 4 head tremor was seen in two patients, which disappeared completely at 12-months. One patient had both facial and voice tremor, with the former disappearing completely and the latter showing marked improvement.

Part B Score

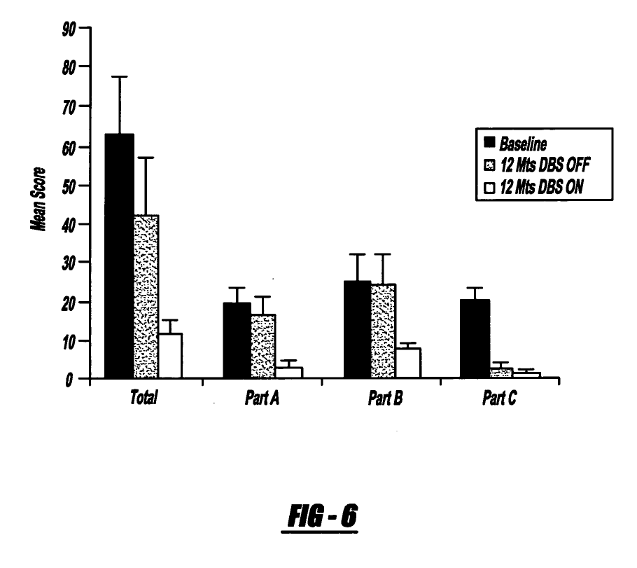

The Part B score (Item 10-14) improved by 67% (baseline mean score of 24.3±7.5 to 8.0±1.2 at 12 months) (FIG. 6). Drawing spirals, drawing lines and pouring water improved significantly (p<0.05) by 66.7%, 58.3% and 76.9% respectively. Handwriting showed a 68% improvement, however this was not found to be a significant change (p>0.05). As the most disabling tremor for patients with ET is postural and action upper limb tremor, we calculated the improvement for this category by summating the postural and action tremor scores of the upper limb with the motor score related to functional activities of the upper limb (writing, drawing and pouring). This category improved by 75.2% following surgery (Table. 1).

Part C Score

Part C score (Item 15-21) improved by 88.8% (mean baseline score 20±3.2 to 2.3±1.5 at 12 months). Individual tasks of daily living (Eating solids, Drinking liquids, Hygiene, Dressing, Writing and Working) also showed marked improvement (Table. 2).

Global Disability Assessment

Before surgery two patients described themselves as being severely disabled (75-100% impairment) and two as markedly disabled (50-75% impairment), based on the global disability assessment (scored by both the patient and the physician) as part of the Clinical Tremor rating score. One year following surgery three had no functional disability and one had mild disability (1-25% impairment).

Medications and Pulse Generator Parameters

Following effective tremor control with DBS, all patients were able to wean off their anti-tremor medication. The mean pulse generator parameters are shown in Table. 3. In all four patients the optimal DBS contact was monopolar stimulation through Contact 2 and 6 as planned preoperatively. There was no significant difference in the settings evaluated at 6 weeks post surgery and at 12 months.

Target verification and Complications

The most effective electrode contact in all the patients was the 3^{rd} contact as planned preoperatively. Peroperative imaging confirmed correct placement of the stylettes to the planned targets except unilaterally in one patient, and this was adjusted appropriately. There were no procedural, device or stimulation related complications.

Discussion This pilot study indicates that medically refractory and disabling ET can be effectively controlled with implantation of bilateral DBS in the subthalamic region.

Surgical Method

The subthalamic area chosen as the optimal target site to control ET is difficult to define with microelectrode recordings as it contains predominantly white matter tracts rather than nuclei. We therefore adopted an image directed method using high resolution and long acquisition T₂ weighted MR scans to identify the target site.

In the immediate vicinity of our planned target are a number of anatomical structures which structures, which could potentially can also influence tremor including the Vim nucleus, Zona Incerta and the subthalamic nucleus. Therefore, accurate identification of the position of the most effective contact was is essential. However, postoperative MR imaging of the DBS electrodes to identify anatomical location of individual contacts is hampered by metal artifact distorting the images. To overcome the above concern we used devised the guide tube method described earlier.

The guide tube with indwelling stylette acts as a device, which enables radiological confirmation of the optimal target localization. Following implantation, the guide tube effectively fixes the brain target and the stylette can be inserted down the guide tube into the target. Peroperative visualisation of the stylette will identify precisely where the DBS lead will subsequently be placed and in turn where each contact will be anatomically positioned. If placement of the stylette is suboptimal this can be identified and the DBS lead position can be adjusted appropriately. This technique is safe and accurate and allows us to perform all functional neurosurgery cases under general anaesthesia.

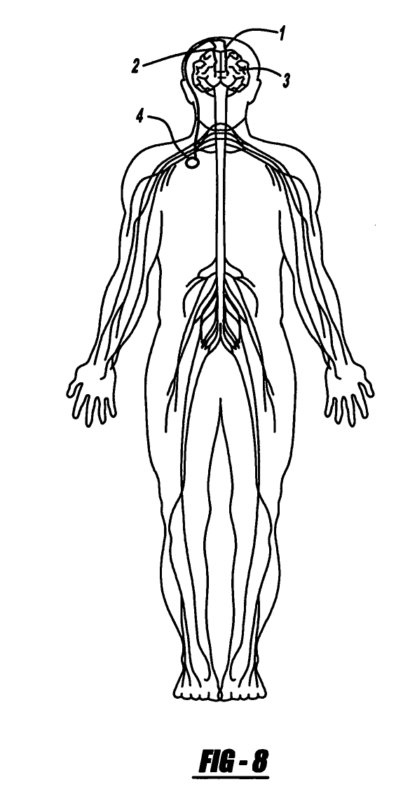

FIG. 8 is a schematic drawing showing a post-operative patient having two electrodes, 1, 2 implanted in the brain, 3. The electrodes are connected to a pulse generator 4 which is implanted under the skin.

Outcome

The total tremor score improved by 80.1%, Part A by 84.2%, Part B 67%, the functional motor score for the upper limb by 75.2% and Part C by 88%. This compares with the multicentre European study in which 37 patients underwent either unilateral or bilateral thalamic stimulation for essential tremor and showed significant (p<0.05) improvements in Part A scores by 55%, Part B by 43.9% and Part C by 80.3%. Pahwa et al in another series of 9 patients with ET, who underwent staged bilateral thalamic stimulation, showed improvements in the total tremor scores by 57%, functional motor scores of the upper limb by 65% and Part C score by 57%. In our series, head and face tremor was completely arrested and marked improvement was noted in voice tremor, when present. In comparison Taha, et al in their series reported a greater than 50% improvement in head tremor in 8 out of 9 patients, with no reported complete arrest. All patients except one underwent bilateral DBS. One patient had multiple sclerosis. Our results based on two patients may suggest that bilateral subthalamic region stimulation can effectively control axial tremor.

Tolerance and Pulse Generator Parameters

Benabid et al. reported tolerance to Vim nucleus stimulation. Tolerance to particular stimulation parameters may occur after days or weeks and a regular increase in stimulation intensity is necessary to maintain control. Even at a maximally tolerable intensity tremor may still breakthrough and in these circumstances it is necessary to stop the stimulation for a variable period (stimulation holiday). Most centres advise turning the stimulator off at night in order to postpone the appearance of tolerance. Benabid et al. found that 18.5% of 22 patients developed tolerance within 3-6 months, with action component of tremor being more susceptible than rest tremor. In our series tolerance was not seen despite maintaining constant stimulation. Excellent tremor control of both the postural and action component was achieved in all patients with complete tremor arrest in ⅝ sides and Grade 1 tremor in ⅜ sides. The original stimulation parameters were not significantly changed and the voltage remained low (mean 1.8□0.1 v). Published reports have shown a higher mean initial voltage which increases with time in order to optimise tremor control, especially the action component.

The inventor attributes the findings of good tremor control at low voltage to the fact that invention targets the ascending dentate/interpositus-thalamic fibres where they are confined to a small volume in the subthalamic region. This is in contrast to the relatively large wedge shaped volume of the Vim nucleus that would be necessary to stimulate in order to achieve the same effect (FIG. 76 ). The low voltage may also be attributed to the fact that axonal tracts are more susceptible to high frequency stimulation than are neuronal bodies as in the Vim nucleus of the thalamus.

Complications

Stimulation related side effects with bilateral Vim DBS include dysarthria and dysequilibrium, reported in up to 30-50% cases in some series. In order to avoid these side effects many have resorted to unilateral or staged bilateral procedures. In our small surgical series, whereby patients underwent 'simultaneous bilateral insertion of DBS, we have had no procedural or stimulation related side effects.

Conclusion

Essential tremor is a fairly common movement disorder, especially in the elderly population. It can be functionally disabling and medically refractory in a high percentage of patients and bilateral Vim stimulation -is associated with a high complication rate. Subthalamic region stimulation deserves further consideration as a potential target for effective control of both distal and axial tremor.

TABLE 1
Pre and post-operative upper limb tremor scores to functional activities.
Data are given as mean ± standard deviation (SD). DBS indicates deep brain stimulation.

| | | Motor | | | | |
|---|---|---|---|---|---|---|
| | | scores | | | | |
| | | related to | | | | |
| | | functional | | | | |
| | | activities | | | | Overall |
| | Postural & | of the | | | | Upper |
| | Action | upper | | | | Limb |
| | component | limb. | Draw | | | Function |
| | tremor score* | Writing | Spiral | Draw Lines | Pour Water | Score# |
| Preoperative | 3.2 ± 0.8 | 2.5 ± 1.0 | 6.0 ± 2.1 | 2.4 ± 1.1 | 2.6 ± 0.9 | 40.3 ± 9.4 |
| DBS OFF 12 | 3.0 ± 0.9 | 2.3 ± 1.0 | 6.0 ± 2.1 | 2.4 ± 1.1 | 2.4 ± 0.9 | 39.0 ± 9.8 |
| Months | | | | | | |
| DBS ON 12 | 0.5 ± 0.5 | 0.8 ± 0.5 | 2.0 ± 0.5 | 1.0 | 0.6 ± 0.5 | 10.0 ± 1.2 |
| Months | | | | | | |
| % Improve | 84.4% | 68% | 66.7% | 58.3% | 76.9% | 75.2% |
| P value | <0.0001 | NS | 0.011 | 0.014 | 0.011 | NS |

Key:
*indicated combined mean postural and action component tremor scores.
#indicates summated score of the action and postural component of tremor and the motor scores related to functional activities of the upper limb.
NS indicates “not significant.” (Number of comparisons only 4)

TABLE 2
Improvement in activities of daily living from baseline and at twelve
month evaluation. Data are given as mean ± standard deviation (SD). DBS
indicates deep brain stimulation ADL indicates activities of daily living.

| | Eating | | | | | | |
|---|---|---|---|---|---|---|---|
| | Solids | Liquids | Hygiene | Dressing | Writing | Working | Overall ADL |
| Preoperative | 3.0 ± 0.8 | 4.0 | 3.5 ± 0.6 | 2.3 ± 1.0 | 3.3 ± 1.0 | 3.0 ± 0.8 | 20 ± 3.2 |
| DBS ON 12 | 0.3 ± 0.5 | 0.5 ± 0.6 | 0 | 0.3 ± 0.5 | 0.5 ± 0.6 | 0.3 ± 0.5 | 2.3 ± 15 |
| months | | | | | | | |
| % Improve | 91.7% | 87.5% | 100% | 89.1% | 84.8% | 91.7% | 88.8% |

| | Amplitude (V) | Frequency (Hz) | Pulse Width (μ sec) |
|---|---|---|---|
| 6 Weeks | 1.8 ± 0.1 | 170 ± 11.5 | 108.8 ± 14.4 |
| 12 Months | 1.8 ± 0.2 | 170 ± 11.5 | 108.8 ± 14.4 |

Throughout this application, various publications, including U.S. patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### REFERENCES

Bain P G, Findley L J, Thompson P D, et al: A study of hereditary essential tremor. Brain 117: 805-824., 1994.
Benabid A L, Pollak P, Gao D, et al: Chronic electrical stimulation of the ventralis intermedius nucleus of the thalamus as a treatment of movement disorders. J Neurosurg,84:203-214., 1996.
Benabid A L, Pollak F, Gervason C, et al: Long-term suppression of tremor by chronic stimulation of the ventral intermediate thalamic nucleus. Lancet 337:403-406., 1991.
Bertrand C, Hardy J,-Molina-Negro P, et al: Optimum physiological target for the arrest of tremor, in Donaldson (ed): 3rd Symposium on Parkinson's Disease: Livingstone, Edinburgh, 1969, pp 251-259.
Blond S, Caparros-Lefebvre D, Parker F, et al: Control of tremor and involuntary movement disorders by chronic stereotactic stimulation of the ventral intermediate thalamic nucleus. J Neurosurg 77:62-68., 1992.
Brin M F, Koller W: Epidemiology and genetics of essential tremor. Mov Disord 13:55-63., 1998.
Constantino A E, Louis E D: Unilateral disappearance of essential tremor after cerebral hemispheric infarct. J Neurol 250:354-355., 2003.
De Zeeuw C I, Simpson J I, Hoogenraad C C, et al: Microcircuitry and function of the inferior olive. Trends Neurosci 21:391-400., 1998.
Deuschl G, Elble R J: The pathophysiology of essential tremor. Neurology 54: S14-20., 2000
Duncan R., Bone I, Melville I D: Essential tremor cured by infarction adjacent to the thalamus. J Neurol Neurosurg Psychiatry 51: 591-592., 1988
Dupuis M J, Delwaide P J, Boucquey D, et al: Homolateral disappearance of essential tremor after cerebellar stroke. Mov Disord 4:183-187., 1989.
Elble R: Central Mechanisms of Tremor. Journal of Clinical Neurophysiology 13:133-144, 1996.
Fahn S, Tolosa E, Marin C: Clinical rating Scale for Tremor., in Jankovic J., Tolosa E (eds): Parkinson's disease and movement disorders. Baltimore Munich: Urban and Schwarenberg, 1988, pp 225-234.
Goldman M S, Ahlskog J E, Kelly P J: The symptomatic and functional outcome of stereotactic thalamotomy for medically intractable essential tremor. J Neurosurg.76: 924-928., 1992.
Hariz M I, Shamsgovara P, Johansson F, et al: Tolerance and tremor rebound following long-term chronic thalamic stimulation for Parkinsonian and essential tremor. Stereotact Funct Neurosurg, 72: 208-218., 1999.
Jankovic J, Cardoso F, Grossman R G, et al: Outcome after stereotactic thalamotomy for parkinsonian, essential, and other types of tremor. Neurosurgery 37:680-686; discussion 686-687., 1995.
Kitagawa M, Murata J, Kikuchi S, et al: Deep brain stimulation of subthalamic area for severe proximal tremor. Neurology 55:114-116., 2000.
Koller W, Pahwa R., Busenbark K, et al: High-frequency unilateral thalamic stimulation in the treatment of essential and parkinsonian tremor. Ann Neurol 42: 292-299., 1997.
Koller W C, Busenbark K, Miner K: The relationship of essential tremor to other movement disorders: report on 678 patients. Essential Tremor Study Group. Ann Neural 35: 717-723., 1994.
Kumar R, Lozano A, Sine E, et al: Delayed failure of thalamic deep brain stimulation (DBS) in Parkinson's disease (PD) and essential tremor (ET). Neurology 52: A457-A458., 1999 (Abstract).
Limousin F, Speelman J D, Gielen F, et al: Multicentre European study of thalamic stimulation in parkinsonian and essential tremor. J Neurol Neurosurg Psychiatry 66:289-296., 1999
Lou J S, Jankovic J: Essential tremor: clinical correlates in 350 patients. Neurology 41: 234-238., 1991.
Mohadjer M, Goerke H, Milios E, et al: Long-term results of stereotaxy in the treatment of essential tremor. Stereotact Funct Neurosurg 54-55:125-129., 1990.
Muarta, et al., J. Neurosurgery 99, 708-715, 2003.
Mundinger F: Stereotaxic interventions on the zona incerta area for treatment of extrapyramidal motor disturbances and their results. Confin Neurol 26: 222-230., 1965.
Nagaratnam N, Kalasabail G: Contralateral abolition of essential tremor following a pontine stroke. J Neurol Sci 149:195-196., 1997.
Nagaseki Y, Shibazaki T, Hirai T, et al: Long-term follow-up results of selective VIM-thalamotomy. J Neurosurg 65:296-302., 1986.
Ondo W, Almaguer M, Jankovic J, et al: Thalamic deep brain stimulation: comparison between unilateral and bilateral placement. Arch Neural 58:218-222., 2001.
Ondo W, Jankovic J, Schwartz K, et al: Unilateral thalamic deep brain stimulation for refractory essential tremor and Parkinson's disease tremor. Neurology 51:1063-1069., 1998.
Pahwa R, Lyons K L, Wilkinson S B, et al: Bilateral thalamic stimulation for the treatment of essential tremor. Neurology 53:1447-1450., 1999
Patel N, Heywood P. O'Sullivan K, et al: MRI-directed subthalamic nucleus surgery for Parkinson's disease. Stereotact Funct Neurosurg 78:132-145., 2002.
Rajput A. H, Rozdilsky B, Ang L, et al: Clinicopathologic observations in essential tremor: report of six cases. Neurology 41:1422-1424., 1991
Ranck J B: Which elements are excited in electrical stimulation of mammalian <central nervous system: a review. Brain Res 98: 417-440, 1975.
Rossitch E Jr, Zeidman S M, Nashold B S Ir, et al: Evaluation of memory and language function pre- and postthalamotoniy with an attempt to define those patients at risk for postoperative dysfunction. Surg Neurol 29:11-16., 1988
Schaltenbrand C: Atlas for Streotaxy of the Human Brain: New York, Georg Thieme, 1977.
Schuurman P R, Bosch D A, Bossuyt P M, et al: A comparison of continuous thalamic stimulation and thalamotomy for suppression of severe tremor. N Eng J Med 342: 461-468., 2000.
Selby G: Stereotactic-surgery for the relief of Parkinson's disease. An analysis of the results in a series of 303 patients (413 operations). J Neurol Sci 5: 343-375, 1967.
Shamsgovara P, Hariz M: Changes of Electrical parameters over time in chronic thalamic stimulation for tremor. Mov Disord 13:73, 1998 (Abstract). Taha J M, Janszen M A, Favre J: Thalamic deep brain stimulation for the treatment of head, voice, and bilateral limb tremor. J Neurosurg 91:68-72., 1999.
Tasker R R, Munz M, Junn F S, et al: Deep brain stimulation and thalamotomy for tremor compared. Acta Neurochir Suppl (Wien) 68: 49-53., 1997.
Velasco F, Velasco M, Machado J P: A statistical outline of the subthalamic target for arrest of tremor. Appl Neurophysiol 38: 28-46., 1975.
Velasco F C, Molina-Negro P, Bertrand C, et al: Further definition of the subthalmic target for arrest of tremor. J Neurosurg 36; 184-191, 1972
Wilms H, Sievers J, Deuschl G: Animal models of tremor. Mov Discord 14: 557-571., 1999.

What is claimed is:

1. A method for treating essential tremor comprising the step of applying deep brain stimulation to the ascending dentate/interpositus-ventral intermedius fibres of the brain at a location remote from the ventral intermedius nucleus of the thalamus.

2. The method according to claim 1, wherein the step of applying deep brain stimulation comprises stimulating the dentate/interpositus-ventral intermedius fibres with an electric field that is sufficiently remote from the sensory thalamus to avoid stimulation of the sensory thalamus.

3. The method according to claim 1, wherein the step of applying deep brain stimulation comprises stimulating the dentate/interpositus-ventral intermedius fibres with an electric field that is remote from synaptic connections of the ventral intermedius nucleus of the thalamus.

4. The method according to claim 1, wherein the step of applying deep brain stimulation comprises the step of introducing an electrode into the brain, such that the electrode is in contact with the dentate/interpositus-ventral intermedius fibres at a location remote from the ventral intermedius nucleus of the thalamus.

5. The method according to claim 4, wherein the step of introducing an electrode into the brain includes locating the electrode substantially at a location identified on the Schaltenbrand Bailey Stereotactic Atlas of the Human Brain, Axial plate 56 LXXVIII H. v 3.5 mm positioned 6.5 mm posterior to the commisural point and 11.5 mm lateral to the anterior/posterior commisural line.

6. The method according to claim 4, further comprising connecting the electrode to a pulse generator.

7. The method according to claim 6, wherein the step of connecting the electrode to a pulse generator includes: providing the electrode on a lead having at least one conductor, and connecting the lead to the pulse generator; the method further comprising implanting the pulse generator in the body of the patient wherein the step of implanting the pulse generator the body of the patient comprises implanting the pulse generator in one of a cranial region or a pectoral region.

8. The method according to claim 7, wherein the step of connecting the lead to the pulse generator includes connecting the lead to the pulse generator with a lead extension; the step of implanting the pulse generator in the body of the patient includes implanting the pulse generator in the pectoral region.

9. The method according to claim 1, wherein the deep brain stimulation is applied bilaterally.

10. The method according to claim 1, wherein the deep brain stimulation is monopolar stimulation.

11. The method according to claim 1, wherein the deep brain stimulation is applied continually.

12. A method for identifying an area of a patient's brain to be targeted with deep brain stimulation for the treatment of essential tremor, comprising the step of using a scan of a patient's brain to identify a target area in relation to the subthalmic nucleus and the red nucleus.

13. The method according to claim 12, wherein the target area is further defined in relation to the zone incerta, the ventral thalamus and the medial lemniscus.

14. The method according to claim 12, wherein the target area is medial to the posterior dorsal third of the subthalmic nucleus.

15. The method according to claim 12, wherein the target area is an area identified on the Shaltenbrand Bailey Stereotactic Atlas of the Human Brain, Axial plate 56 LXXVIII H. v-3.5 mm positioned 6.5 mm posterior to the intercommisural point and 11.5 mm lateral to the anterior/posterior commisural line.

16. The method according to claim 12, wherein the scan is an MR scan.

17. The method according to claim 16, wherein the scan is a T₂ weighted MR scan.

18. A method of treating essential tremor by using deep brain stimulation of the dentate/interpositus-ventral intermedius fibres of the brain at a location remote from the ventral intermediate nucleus of the thalamus in the treatment of essential tremor.

19. The method according to claim 18, wherein the deep brain stimulation of dentate/interpositus-ventral intermedius fibres comprises stimulation of the dentate-thalamic fibres with an electric field that is sufficiently remote from the sensory thalamus to avoid stimulation of the sensory thalamus.

20. The method according to claim 18, wherein deep brain stimulation of dentate-thalamic fibres comprises stimulation of the dentate-thalamic fibres with an electric field that is remote from synaptic connections of the ventral intermediate nucleus of the thalamus.

21. A method of treating essential tremor by using a DBS electrode targeted to the dentate/interpositus-ventral intermedius fibres at a location remote from the ventral intermedius nucleus of the thalamus in the preparation of a component for the treatment of essential tremor.

22. A kit for use in treating essential tremor comprising a DBS electrode and instructions for how to identify dentate/interpositus-ventral intermedius fibres at a location remote from the ventral intermedius nucleus of the thalamus.

23. The kit according to claim 22, further comprising instructions for identifying a location remote from the ventral intermedius nucleus of the thalamus.

24. The kit according to claim 23 further comprising instructions for how to position the DBS electrode during treatment.
